(19) United States
(12) Patent Application Publication
(10) Pub. No.: US 2017/0327868 A1
(43) Pub. Date: Nov. 16, 2017

(54) BLOCKER BASED ENRICHMENT SYSTEM AND USES THEREOF

(71) Applicant: Admera Health LLC, South Plainfield, NJ (US)

(72) Inventors: Jun Huang, South Plainfield, NJ (US); Danielle Quintanilha, South Plainfield, NJ (US); Guanghui Hu, South Plainfield, NJ (US); Yang Song, South Plainfield, NJ (US)

(73) Assignee: Admera Health LLC, South Plainfield, NJ (US)

(21) Appl. No.: 15/514,852

(22) PCT Filed: Oct. 20, 2016
(86) PCT No.: PCT/US16/57805

§ 371 Date: Mar. 28, 2017

Related U.S. Application Data
(60) Provisional application No. 62/244,279, filed on Oct. 21, 2015.

(51) Int. Cl. C12Q 1/68; C12Q 1/00

(52) U.S. Cl. CPC C12Q 1/6806; C12Q 1/686; C12Q 1/00

(57) ABSTRACT

The present invention relates to a PCR-based systems and methods for enrichment of minority alleles and mutations.

### CROSS REFERENCE TO RELATED APPLICATION

This application claims priority to U.S. Provisional Application No. 62/244,279 filed on Oct. 21, 2015. The content of the application is incorporated herein by reference in its entirety.

### FIELD OF THE INVENTION

This invention relates to PCR-based systems and methods for enrichment of minority alleles and mutations.

### BACKGROUND OF THE INVENTION

Detection of nucleic acid mutations and variants is important for various situations, including detection and prognosis of diseases. A prominent concern confronting clinical and diagnostic applications is the ability to detect clinically significant low-level mutations and minority alleles. The ability to discern mutations is important in many regards, but especially for early cancer detection from tissue biopsies and bodily fluids such as plasma or serum; assessment of residual disease after surgery or radio/chemotherapy; disease staging and molecular profiling for prognosis or tailoring therapy to individual patients; and monitoring of therapy outcome and cancer remission/relapse. Efficient detection of cancer-relevant somatic mutations largely depends on the selectivity of the techniques and methods employed.

Detection and identification of oncogene and tumor-suppressor gene mutations primarily require analysis of precancerous or cancerous tissue, sputum, urine, stool, and circulating extracellular DNA released in blood. The sample is typically composed of both wild-type and mutant DNA, and the quantity of wild type DNA often exceeds the mutant DNA contribution. In many cases, wild-type DNA vastly exceeds mutant DNA, making it difficult to detect and identify minority alleles present at extremely low concentrations. Thus, there is a need for systems and methods for enrichment of minority alleles and mutations.

### SUMMARY OF INVENTION

This invention addresses the above-mentioned need by providing systems and methods for enrichment of minority alleles and mutations.

In one aspect, the invention provides a set of nucleic acid molecules for enriching an allelic variant in a target sequence of a target nucleic acid, comprising (a) a forward primer and a reverse primer that are capable of amplifying the target sequence and (b) a first blocker comprising a first sequence that (i) is matched or complementary to the wild-type allele in the target sequence and (ii) is capable of being extended by a DNA polymerase. The set of nucleic acid molecules can further comprise a second blocker having a second sequence that is matched or complementary to the complement of the wild-type allele.

In some embodiments, the first or second blocker does not overlap with either the forward primer or the reverse primer. In some other embodiments, the first or second blocker can contain one or more modified nucleic acids or linkages. The first or second blocker can also have a modified nucleic acid or linkage at the 3′ end. The modified nucleotides or linkages can comprise PNA, LNA, a 2′-O-Methyl nucleic acid, a 2′-O-Alkyl nucleic acid, a 2′-fluoro nucleic acid, a phosphorothioate linkage, and any combination thereof.

In some examples, the target sequence spans a region encoding EGFR T790M, EGFR L858R, BRAF V600E, BRAF V600K, BRAF V600D, BRAF V600G, BRAF V600A, BRAF V600R, or one selected from a group consisting of those listed in Tables 8 and 9 below.

The forward or reverse primer can be about 10-50, e.g., about 15-30, about 16-28, about 17-26, about 18-24, or about 20-22 nucleotides in length. The first or second blocker can be about 5-100 nucleotides length, e.g., about 10-50, about 15-30, about 16-28, about 17-26, about 18-24, or about 20-22 nucleotides in length. In preferred embodiments, the blockers in general are shorter than the primers.

The invention also provides a kit comprising the set of nucleic acid molecules described above and one or more reagents for conducting an amplification reaction. The kit can comprise one or more reagents selected from the group consisting of a buffer, a DNA polymerase, an RNAse inhibitor, extension nucleotides, and a probe.

Also provided is a method for enriching an allelic variant in a target sequence. The method includes providing the set of nucleic acid molecules described above; amplifying the target sequence with the forward primer and the reverse primer in the presence of the first or second blocker.

The enriching method can be used in a method for detecting the presence or absence of an allelic variant in a target sequence. This method includes enriching the allelic variant as described above to generate an amplification product; and examining the amplification product for the presence or absence of the allelic variant. The examining step can be carried out by gene sequencing, qPCR, or any other techniques known in the art. The target sequence can span a region encoding EGFR T790M, EGFR L858R, BRAF V600E, BRAF V600K, BRAF V600D, BRAF V600G, BRAF V600A, BRAF V600R, or one selected from a group consisting of those listed in Tables 8 and 9.

The enriching method described above can also be used in a method for evaluating a subject having cancer or suspected of having cancer (e.g., lung cancer and melanoma). Such an evaluation method includes obtaining a biological sample from the subject; and performing an assay to determine the presence or absence of one or more allelic variants in the biological sample as described above. In one example, the cancer is lung adenocarcinoma, such as non-small cell lung cancer. The biological sample can be serum, plasma, whole blood, saliva, or sputum. The method can further include determining or recommending a treatment course of action based on the presence of said one or more allelic variants. The method can also include a step of administering said treatment when said one or more allelic variants are present.

The details of one or more embodiments of the invention are set forth in the description below. Other features, objectives, and advantages of the invention will be apparent from the description and from the claims.

### BRIEF DESCRIPTION OF THE DRAWINGS

FIGS. 1A, 1B, and 1C are diagrams showing allele-specific PCR (AS-PCR) using a mutant specific oligo. FIG. 1A: the oligo is perfectly matched to mutant sequences and allows extension and PCR amplification with a reverse primer (not shown). FIG. 1B: the oligo has a mismatch at 3′ end and does not match wild type sequence, thus prevents the oligo extension and amplification. FIG. 1C: non-specific extension does occur and results in false positive PCR products containing mutant allele sequence.

FIGS. 2A, 2B, and 2C are diagrams showing an exemplary system of this invention: FIG. 2A: A blocker anneals to a wild type allele and the extended blocker blocks PCR amplification of the outer primers. FIG. 2B: Each blocker oligo has a mismatch at 3′ end and does not stay annealed to mutant allele, thus allows amplification by outer primers. FIG. 2C: Non-specific extension only results in amplification failure of the particular template allele in this singular cycle. No false positive PCR product is formed.

### DETAILED DESCRIPTION OF THE INVENTION

This invention is based, at least in part, on an unexpected discovery that that oligonucleotide blockers that are matched to or complementary to a particular nucleic acid variant (such as a wild type variant) can block or suppress the amplification of that particular nucleic acid variant, thereby allowing enrichment of other variants (e.g., mutant variants). Accordingly, the present invention provides enrichment reaction systems and methods for detecting the presence or absence of a nucleic acid variant in a target region.

### Systems

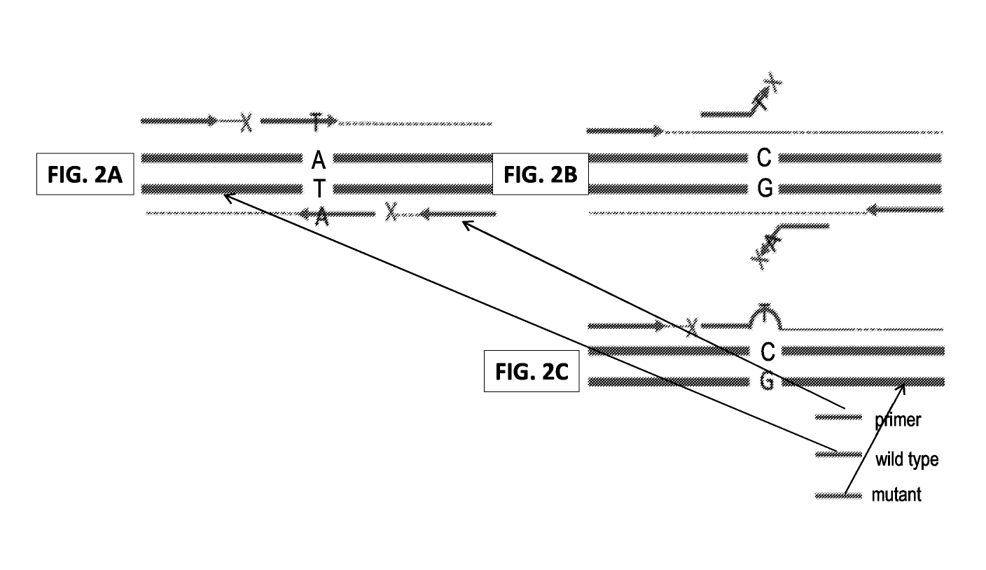

In some embodiments, an enrichment reaction system of this invention comprises primers, blockers, and essential ingredients for PCR amplification. As shown in FIGS. 2A-C, the system of this invention can have (i) a first blocker that binds or hybridizes to the same strand or sequence as the forward primer and (ii) a the second blocker and a reverse primer bind or hybridize to the opposite strand and/or complementary sequence. A primer pair (i.e., a forward primer and a reverse primer) is used to amplify the region containing hotspot mutation and blockers are used to block the amplification of a nucleic acid variant (e.g., an abundant allelic variant such as a wild type allele). A blocker is an oligo complementary to the nucleic acid variant (e.g., wild type allele). Its 3′ end is designed to match perfectly to that variant of interest. For example, it perfectly anneals to the wile type allele and is able to be extended by a DNA polymerase. Melting temperature is highly correlated with the length of an oligo. By extending the length of the blocker oligo, the blocker withstands a higher reaction temperature and thus stays associated with the wild type allele. On the other hand, at an initial low reaction temperature, the blocker can also anneal to mutant allele; however, because the mutated bases of the mutant allele do not match the 3′ end of the blocker, extension does not occur. This results in dissociation of the blocker from mutant allele once temperature rises. Therefore, the blocker is tightly annealed to wild type while is easily denatured from mutant. Primers are in the reaction to amplify the region of interest. Since the blocking oligo is annealed to wild type, the primer extension cannot continue pass the blocked region, and thus mutant allele is preferentially amplified.

For example, as shown in FIG. 2A, the blocker anneals to a wild type allele and the extended oligo blocker blocks PCR amplification of the outer primers. In FIG. 2B, each oligo blocker has a mismatch at 3′ end and does not stay annealed to mutant allele, thus allows amplification by outer primers. In FIG. 2C, a non-specific extension only results in amplification failure of the particular template allele in this singular cycle. No false positive PCR product is formed.

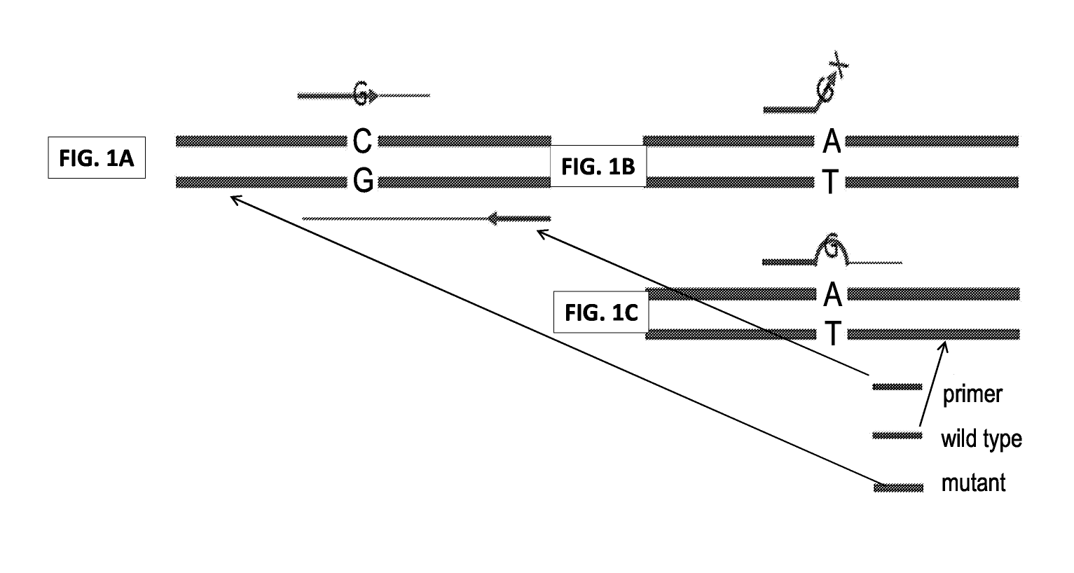

The system disclosed herein is superior to allele-specific PCR (AS-PCR), also known as amplification mutation refractory system (Newton C et al., Nucleic Acids Res. 1989 Apr. 11; 17(7): 2503-2516), which is a tried-and-true technique to enrich hotspot mutations. In the AS-PCR approach, the 3′ end of the primers is designed to match perfectly to a variant of interest and allow specific mutant amplification (FIGS. 1A and 1B). Qiagen Therascreen EGFR and Roche Cobas EGFR systems, for instance, adopted this technique. However, the inherent disadvantage of non-specific extension of the allele-specific primer (FIG. 1C) lowers sensitivity that leads to unreliable discrimination between rare somatic mutant and wild type. LOD is documented 0.5%-7.02% for Therascreen and 5% for Cobas.

### Blocker

As mentioned above, a blocker (herein sometimes referred to as “blocking oligo”) is complementary to a particular nucleic acid variant to be suppressed, such as abundant allelic variant (e.g., a wild type allele). Such a blocker may be designed as short oligomers that are single-stranded and have a length of 100 nucleotides or less, more preferably 50 nucleotides or less, still more preferably 30 nucleotides or less and most preferably 20 nucleotides or less with a lower limit being approximately 5 nucleotides.

The blocker can in some cases be modified by a variety of methods known in the art to protect against 3′ or 5′ exonuclease activity. The blocker can include one or more modifications to protect against 3′ or 5′ exonuclease activity and such modifications can include but are not limited to 2′-O-methyl ribonucleotide modifications, phosphorothioate backbone modifications, phosphorodithioate backbone modifications, phosphoramidate backbone modifications, methylphosphonate backbone modifications, 3′ terminal phosphate modifications and 3′ alkyl substitutions. In some embodiments, the blocker is resistant to 3′ and/or 5′ exonuclease activity due to the presence of one or more modifications.

Its 3′ end is designed to match perfectly to a particular nucleic acid variant of interest. As shown in the examples below, a blocker perfectly anneals to the wile type allele and is able to be extended by a DNA polymerase.

Melting temperature (Tm) of the blocker is highly correlated with its length. The Tm of the blocker can range from 40° C. to 70° C., such as 40° C. to 70° C., 41° C. to 69° C., 42° C. to 68° C., 43° C. to 67° C., 44° C. to 66° C., or about 530° C. to about 56° C., or any range in between. In yet other embodiments, the Tm of the blocker can be about 3° C. to 6° C. higher than the anneal/extend temperature in the PCR cycling conditions employed during amplification.

In some embodiments, the blocker is not cleaved during PCR amplification. According to the present invention, the blocker can be either extendable or non-extendable. In some embodiments, the blocker can comprise a non-extendable blocker moiety at its 3′-end. In some embodiments, the blocker can further comprise other moieties (including, but not limited to additional non-extendable blocker moieties, quencher moieties, fluorescent moieties, etc.) at its 3′-end, 5′-end, and/or any internal position in between. In others, the blocker is extendable and does not contain any non-extendable blocker moiety at its 3′-end. In that case, the blocker is extended during PCR. By extending the length of the blocking oligo, the blocker withstands a higher reaction temperature and thus stays associated with the wild type allele.

### Primers

According to the present invention, a forward primer and/or reverse primer can be designed to be complementary (fully or partially) to various suitable positions relative to one or more nucleic acid variants of interest. For example, the 3′ region of the forward primer or reverse primer when hybridized to the target region in some cases can be located 0, 5, 10, 15, 20, 25, 30, 35, 40, 45, 50, 55, 60, 80, 100, 250, 500, 1000, 2000 or more nucleotides away from one or more nucleic acid variants in the target region. In some embodiments, the 3′ region of the forward primer or reverse primer when hybridized to the target region is located less than about 30 nucleotides away from one or more nucleic acid variants in the target region. The primers can be oligomers ranging from about 10-50, e.g., about 15-30, about 16-28, about 17-26, about 18-24, or about 20-22, or any range in between, nucleotides in length.

In some instances, the forward or reverse primer and blockers can overlap and compete for hybridizing to a partial or full target region. For example, the primer and blockers can overlap by 0, 5, 10, 15, or more nucleotides. In some embodiments, the primer and blockers do not overlap or compete for hybridizing to a partial or full target region at all.

The above-discussed primers and/or the blockers can comprise one or more modified nucleobases or nucleosidic bases different from the naturally occurring bases (i.e., adenine, cytosine, guanine, thymine and uracil). In some embodiments, the modified bases are still able to effectively hybridize to nucleic acid units that contain adenine, guanine, cytosine, uracil or thymine moieties. In some embodiments, the modified base(s) may increase the difference in the Tm between matched and mismatched target sequences and/or decrease mismatch priming efficiency, thereby improving not only assay specificity, bust also selectivity.

Modified bases are considered to be those that differ from the naturally-occurring bases by addition or deletion of one or more functional groups, differences in the heterocyclic ring structure (i.e., substitution of carbon for a heteroatom, or vice versa), and/or attachment of one or more linker arm structures to the base. In some embodiments, all tautomeric forms of naturally occurring bases, modified bases and base analogues may also be included in the oligonucleotide primers and blockers of the invention.

Some examples of modified base(s) may include, for example, the general class of base analogues 7-deazapurines and their derivatives and pyrazolopyrimidines and their derivatives (see e.g., WO 90/14353 and US20100285478, the content which are incorporated herein by reference in their entireties). Examples of base analogues of this type include, for example, the guanine analogue 6-amino-1H-pyrazolo[3,4-d]pyrimidin-4(5H)-one (ppG), the adenine analogue 4-amino-1H-pyrazolo[3,4-d]pyrimidine (ppA), and the xanthine analogue 1H-pyrazolo[4,4-d]pyrimidin-4(5H)-6(7H)-dione (ppX). These base analogues, when present in an oligonucleotide of some embodiments of this invention, strengthen hybridization and can improve mismatch discrimination.

Additionally, in some embodiments, modified sugars or sugar analogues can be present in one or more of the nucleotide subunits of an oligonucleotide in accordance with the invention. Sugar modifications include, but are not limited to, attachment of substituents to the 2′, 3′ and/or 4′ carbon atom of the sugar, different epimeric forms of the sugar, differences in the α or β-configuration of the glycosidic bond, and other anomeric changes. Sugar moieties include, but are not limited to, pentose, deoxypentose, hexose, deoxyhexose, ribose, deoxyribose, glucose, arabinose, pentofuranose, xylose, lyxose, and cyclopentyl.

Locked nucleic acid (LNA)-type modifications, for example, typically involve alterations to the pentose sugar of ribo- and deoxyribonucleotides that constrains, or “locks,” the sugar in the N-type conformation seen in A-form DNA. In some embodiments, this lock can be achieved via a 2′-O, 4′-C methylene linkage in 1,2:5,6-di-O-isopropylene-α-D-allofuranose. In other embodiments, this alteration then serves as the foundation for synthesizing locked nucleotide phosphoramidite monomers. (See, for example, Wengel J., Ace. Chem. Res., 32:301-310 (1998), U.S. Pat. No. 7,060,809; Obika, et al., Tetrahedron Lett 39: 5401-5405 (1998); Singh, et al., Chem Commun 4:455-456 (1998); Koshkin, et al., Tetrahedron 54: 3607-3630 (1998), the disclosures of each of which are incorporated herein by reference in their entireties.)

In some preferred embodiments, the modified bases include 8-Aza-7-deaza-dA (ppA), 8-Aza-7-deaza-dG (ppG), 2′-Deoxypseudoisocytidine (iso dC), 5-fluoro-2′-deoxyuridine (fdU), locked nucleic acid (LNA), or 2′-0,4′-C-ethylene bridged nucleic acid (ENA) bases. Other examples of modified bases that can be used in the invention are described in U.S. Pat. No. 7,517,978, the disclosure of which is incorporated herein by reference in its entirety.

Many modified bases, including for example, LNA, ppA, ppG, 5-Fluoro-dU (fdU), are commercially available and can be used in oligonucleotide synthesis methods well known in the art. In some embodiments, synthesis of modified primers and blockers can be carried out using standard chemical means also well known in the art. For example, in certain embodiments, the modified moiety or base can be introduced by use of a (a) modified nucleoside as a DNA synthesis support, (b) modified nucleoside as a phosphoramidite, (c) reagent during DNA synthesis (e.g., benzylamine treatment of a convertible amidite when incorporated into a DNA sequence), or (d) by post-synthetic modification.

Due to the flexibility of the nucleotide structure, some mismatched base pairs can partially conform for Watson-Crick binding. This feature renders the blocker inefficient as it can also be extended on mutant allele. Locked Nucleic Acid (LNA) and some other nucleic acid analogues with a more rigid structure can be used to alleviate the problem. The blockers used can contain one or two LNA at and/or near the variant of interest.

In some embodiments, the primers or blockers are synthesized so that the modified bases are positioned at the 3′ end. In some embodiments, the modified base are located between, 1-6 nucleotides, e.g., 2, 3, 4 or 5 nucleotides away from the 3′-end of the primer or blocker. In some preferred embodiments, the primers or blockers are synthesized so that the modified bases are positioned at the 3′-most end.

Modified internucleotide linkages can also be present in primers and blockers disclosed in this invention. Such modified linkages include, but are not limited to, peptide, phosphate, phosphodiester, alkylphosphate, alkanephosphonate, thiophosphate, phosphorothioate, phosphorodithioate, methylphosphonate, phosphoramidate, substituted phosphoramidate and the like. Several further modifications of bases, sugars and/or internucleotide linkages, that are compatible with their use in oligonucleotides serving as blockers and/or primers, will be apparent to those of skill in the art.

DNA polymerase with 3′ to 5′ exonuclease activity is able to remove mismatched bases from 3′ end of an oligo, and thus the discriminating bases from the blockers can be removed. A phosphorothioate bond is more resistant to exonuclease activity than the indigenous phosphodiester bond; therefore it is used at the 3′ of the blockers.

### Methods

The above-described system can be used in various methods for identifying, enriching, and/or quantifying an allele variant in a sample.

A method disclosed in the invention generally includes amplifying a target region with a forward primer and a reverse primer in the presence of one or more blockers. The blocker includes a sequence complementary to the target region in the absence of the nucleic acid variant to be enriched. The methods can further include detecting amplification of the target region. The methods of the present invention allow one to detect nucleic acid variants with very high sensitivity, in some cases at a limit of detection (LOD) of about 0.01% to 0.001%.

As shown in FIG. 2A, when a blocker anneals to a variant (e.g., a wild type variant), the blocker extends and blocks PCR amplification, thereby preventing the extension of a distant forward primer or reverse primer. In some embodiments, the forward or reverse primer can be located 0, 5, 10, 15, 20, 25, 30, 35, 40, 45, 50, 55, 60, 80, 100, 250, 500, 1000, 2000 or more nucleotides away from the region where the blocker hybridize. In some embodiments, the blocker has a sufficiently high Tm that it is not displaced by a replicating forward primer or reverse primer. In some embodiments, the enzyme used during the amplification reaction does not comprise a strand displacement activity.

Various DNA polymerases can be used in this invention. In some cases, the enzymes employed with the methods of the present invention for amplification of the target region include but are not limited to high-fidelity DNA polymerases and repair enzymes that possess 3′ exonuclease repair activity. Exemplary enzymes for use with the methods of the invention can include but are not limited, Pfu Turbo Hotstart DNA Polymerase, Phusion Hot Start High Fidelity DNA Polymerase, Phusion Hot Start II High Fidelity DNA Polymerase, Phire™ Hot Start DNA Polymerase, Phire™ Hot Start II DNA Polymerase, KOD Hot Start DNA Polymerase, Q5 High Fidelity Hot Start DNA Polymerase, AmpliTaq, Phusion HS II, Deep Vent, and Kapa HiFi DNA polymerase.

General methods for amplifying nucleic acid sequences are well known in the art. Any such methods can be employed with the methods of the present invention. In some embodiments, the amplification uses digital PCR methods, such as those described, for example, in Vogelstein and Kinzler (“Digital PCR,” PNAS, 96:9236-9241 (1999); incorporated by reference herein in its entirety). Such methods include diluting the sample containing the target region prior to amplification of the target region. Dilution can include dilution into conventional plates, multiwell plates, nanowells, as well as dilution onto micropads or as microdroplets. (See, e.g., Beer N R, et al., “On-chip, real-time, single-copy polymerase chain reaction in picoliter droplets,” Anal. Chem. 79(22):8471-8475 (2007); Vogelstein and Kinzler, “Digital PCR,” PNAS, 96:9236-9241 (1999); and Pohl and Shih, “Principle and applications of digital PCR,” Expert Review of Molecular Diagnostics, 4(1):41-47 (2004); all of which are incorporated by reference herein in their entirety.). When combined with digital PCR, the present invention can greatly increase the sensitivity of digital PCR. This is due in part to the fact that the current invention provides methods for significantly suppressing (e.g., at least about 60%, 65%, 70%, 75%, 80%, 85%, 90%, 95%, 97%, 98%, 99%, 99.9%, 99.99%, 99.999%, or about 100%) wild-type associated background, when interrogating genetic events, including for example rare genetic events. The sensitivity of targeting provided by the methods of the present invention allows far higher target loading in the individual volume elements of the single digital PCR reactions.

When running PCR (e.g., digital PCR) for detecting rare genetic events, most of the events present in a given reaction mixture will be of a wild-type sequence while very few will contain the rare genetic event. The methods of the present invention provides for very effective wild-type suppression, for example greater than 1:10,000 as described herein. In some embodiments, 10,000 wild-type targets can be present in each PCR digital element while still allowing for detection of a single rare target due to the effective suppression of the wild-type amplification combined with not suppressing amplification of the single rare target.

The methods of the present invention can further include detecting amplification of the target region using any detection method well known in the art. For example, detection can be by obtaining melting curves for the amplified products, by mass spectrometry, or by sequencing of the amplified products. Amplification products will exhibit different melting curves depending on the type and number of nucleic acid variants in the amplification product. Methods for determining melting curves have been well described and are well known to those of skill in the art and any such methods for determining melting curves can be employed with the methods of the present invention. Methods for the use of mass spectrometry as well as methods for sequencing nucleic acids are also all well known in the art. (See, for e.g., Sambrook and Russell, Molecular Cloning: A Laboratory Manual, (3^{rd} ed.) (2001) and Plum, Optical Methods, Current Protocols in Nucleic Acid Chemistry, 2001-2011); all of which are incorporated by reference herein in their entirety). (See, for e.g., Current Protocols in Nucleic Acid Chemistry, 2001-2011, specifically Liquid Chromatography-Mass Spectrometry Analysis of DNA Polymerase Reaction Products; incorporated by reference herein in its entirety.) Methods for nucleic acid sequencing are also routine and well known by those skilled in the art and any methods for sequencing can be employed with the methods of the present invention. (See, e.g., Current Protocols in Molecular Biology, 1995-2010; incorporated by reference herein in its entirety.)

The methods of the invention further include detecting amplification of the target region by comparing the quantity of the amplified product to a predetermined level associated with the presence or absence of the nucleic acid variant in the target region. Methods for detecting amplification or determining the quantity of an amplified product are well known in the art and any such methods can be employed. (See, e.g., Sambrook and Russell, Molecular Cloning: A Laboratory Manual (3^{rd} ed.) (2001) and Gallagher, Current Protocols Essential Laboratory Techniques, 2008); all of which are incorporated by reference herein in their entirety.)

The nucleic acid variants that can be detected by the methods of the present invention include mutations in the target region, deletions in the target region, and/or insertions in the target region. Deletions include removal of a nucleotide base from the target region. Deletions that can be detected include deletion of 1, 2, 3, 4, 5 or more (such as hundreds of or thousands of) nucleotide bases from the target region. Mutations can include but are not limited to substitutions (such as transversions and transitions), abasic sites, crosslinked sites, and chemically altered or modified bases. Mutations that can be detected include mutation of 1, 2, 3, 4, 5 or more nucleotide bases within the target region. Insertions include the addition of a nucleotide into a target region. Insertions that can be detected can include insertion of 1, 2, 3, 4, 5 or more (such as hundreds of or thousands) nucleotide bases into the target region. In some embodiments, a deletion, a mutation and/or an insertion is detected by the methods of the present invention.

The system and method disclosed herein minimize false positives, the biggest weakness of AS-PCR. Instead of extending mutant DNA, the blocking oligos anneal to the wild type DNA and block its amplification and, consequently, the mutant DNA is preferentially amplified (FIGS. 2A and 2B). The occasional allelic non-specific blocker extension has nearly no effect on enrichment outcome (FIG. 2C). Amplification of the residual non-blocked wild type can be recognized in subsequent NGS sequencing.

As disclosed herein, the system and method described in this invention efficiently block more than e.g., 99.9% of wild type amplification and result in the significantly augmented presence of a mutant allele from one in ten thousand up to one in seven, that is from about 0.01% to about 14%. Rare false positives are not introduced by the intrinsic non-specific extension of allele-specific oligos but rather only result from the nucleotide incorporation errors by DNA polymerase. Using high-fidelity polymerase with 3′ to 5′ exonuclease activity further reduces the rare false positives.

### Uses and Applications

The method disclosed herein can be used for enriching or detecting various target nucleic acid of interest. The target nucleic acid can be a part of a double stranded nucleic acid or a single-stranded nucleic acid.

Sources of nucleic acid samples that can be used include, but are not limited to, human cells such as circulating blood, cultured cells and tumor cells. Also other mammalian tissue, blood and cultured cells are suitable sources of template nucleic acids. In addition, viruses, bacteriophage, bacteria, fungi and other micro-organisms can be the source of nucleic acid for analysis. The DNA may be genomic or it may be cloned in plasmids, bacteriophage, bacterial artificial chromosomes (BACs), yeast artificial chromosomes (YACs) or other vectors. RNA may be isolated directly from the relevant cells or it may be produced by in vitro priming from a suitable RNA promoter or by in vitro transcription. The present invention may be used for the detection of variation in genomic DNA whether human, animal or other. It finds particular use in the analysis of inherited or acquired diseases or disorders. A particular use is in the detection of inherited diseases and cancer.

In some embodiments, a template sequence or nucleic acid sample can be genomic DNA. In other embodiments, the template sequence or nucleic acid sample can be cDNA. In yet other embodiments, as in the case of simultaneous analysis of gene expression by RT-PCR, the template sequence or nucleic acid sample can be RNA. The DNA or RNA template sequence or nucleic acid sample can be extracted from any type of tissue including, for example, formalin-fixed paraffin-embedded tumor specimens.

In some embodiments, the target nucleic acid strand can be one present in a cell of a subject, such as a mammal (e.g., human), a plant, a fungus (e.g., a yeast), a protozoa, a bacterium, or a virus. For example, the target nucleic acid can be present in the genome of an organism of interest (e.g., on a chromosome) or on an extrachromosomal nucleic acid. In some embodiments, the target nucleic acid can be RNA, e.g., an mRNA. In some other embodiments, the target nucleic acid can be DNA (e.g., double-stranded DNA).

In particular embodiments, the target nucleic acid can be specific for the organism of interest, i.e., the target nucleic acid is not found in other organisms or not found in organisms similar to the organism of interest. In some embodiments, the target nucleic acid can be a viral nucleic acid. For example, the viral nucleic acid can be that in human immunodeficiency virus (HIV), an influenza virus (e.g., an influenza A virus, an influenza B virus, or an influenza C virus), or a dengue virus. The target nucleic acid can be present in a bacterium. In some embodiments, the target nucleic acid can be a protozoan nucleic acid. In some embodiments, the target nucleic acid is a fungal (e.g., yeast) nucleic acid.

In some preferred embodiments, the target nucleic acid can be a mammalian (e.g., human) nucleic acid. For example, the mammalian nucleic acid can be found in circulating tumor cells, epithelial cells, or fibroblasts. In other examples, the target nucleic acid is nucleic acids circulating freely in the blood of a subject, such as cell free nucleic acids (cfNA) or circulating tumor DNA (ctDNA) in the blood of a cancer patient. To best utilize the limited amount of cell free nucleic acids (cfNA) obtained from patient plasma, multiplex PCR chemistry and a universal PCR reaction program can be used. As shown in the Examples below, results show successful co-enrichment of all three tested mutation hotspots. Multiplex assay allows simultaneous examination of an array of driver mutations, a requirement for a comprehensive lung cancer liquid biopsy.

To reach the goal of 5-day turnaround time recommended by CAP (Lindeman N I et al., Arch Pathol Lab Med. 2013; 137(6):828-860. doi:10.5858/arpa.2012-0720-OA), the library preparation procedure was optimized to under 4 hours. It is estimated that the turnaround time for the liquid biopsy is 4 days taking in account of sample logistics, sequencing, data analysis and interpretation.

In one example, the target strand is one containing a particular variant, such as single-nucleotide polymorphism (SNP) or a genetic mutation. Examples of such a mutation include a point mutation, a translocation, or an inversion.

In some embodiments, the compositions, methods, and/or kits disclosed herein can be used in detecting circulating cells in diagnosis. In one embodiment, the compositions, methods, and/or kits can be used to detect tumor cell DNA in blood for early cancer diagnosis. In some embodiments, the compositions, methods, and/or kits can be used for cancer or disease-associated genetic variation or somatic mutation detection and validation. In some embodiments, the compositions, methods, and/or kits can be used for genotyping tera-, tri- and di-allelic SNPs. In other embodiments, the compositions, methods, and/or kits can be used for identifying single or multiple nucleotide insertion or deletion mutations. In some embodiments, the compositions, methods, and/or kits can be used for DNA typing from mixed DNA samples for QC and human identification assays, cell line QC for cell contaminations, allelic gene expression analysis, virus typing/rare pathogen detection, mutation detection from pooled samples, detection of circulating tumor cells in blood, and/or prenatal diagnostics.

### Cancer-Related Uses

The system and method disclosed in this invention are particularly useful in the areas of (a) early cancer detection from tissue biopsies and bodily fluids such as plasma or serum; (b) assessment of residual disease after surgery or radiochemotherapy; (c) disease staging and molecular profiling for prognosis or tailoring therapy to individual patients; and (d) monitoring of therapy outcome and cancer remission/relapse.

Cancer deaths in the United States are projected at 589,430 in 2015 (Siegel R L et al. CA Cancer J Clin. 2015; 65(1):5-29. doi:10.3322/caac.21254). Among the cancers, non-small cell lung cancer (NSCLC), the number one cause of cancer mortality, accounts for 22.8% (Siegel R L et al. CA Cancer J Clin. 2015; 65(1):5-29. doi:10.3322/caac.21254.). Platinum-based combination chemotherapy moderately improves advanced NSCLC patient survival by 9% at 12 months compared to supportive care alone (Spiro S G et al. Thorax. 2004; 59(10): 828-836. doi: 10.1136/thx.2003.020164.). In comparison, targeted therapy incorporates tumor genotyping into therapeutic decision-making and has greatly improved upon treatment efficacy. For instance, gefitinib, a small-molecule tyrosine kinase inhibitor (TKI) that targets epidermal growth factor receptor (EGFR) (Paez J G et al., EGFR Mutations in Lung Cancer: Correlation with Clinical Response to Gefitinib Therapy. 2004; 304(June):1497-1501.), has a response rate up to 75% compared to 20% by typical chemotherapy and median survival of 10.3 months versus 28.2 months (Barr Kumarakulasinghe N, et al. Respirology. February 2015. doi: 10.1111/resp.12490.). However, targeted therapy does not benefit all NSCLC patients. The high efficacy relies on the existence of actionable oncogenic driver mutations in patients. These mutations are molecular abnormalities that initiate or maintain the neoplastic process and can be negated by agents directed against each genomic alteration. Without targetable neoplastic process, gefitinib has a miniscule 0-6.6% response rate in NSCLC patients with a wild type EGFR gene, but an exceptional 75% response rate in patients with EGFR mutations (Douillard J-Y, et al. J Clin Oncol. 2010; 28(5):744-752. doi:10.1200/JCO.2009.24.3030; Hirsch F R et al. J Clin Oncol. 2006; 24(31):5034-5042. doi:10.1200/JCO.2006.06.3958; Maruyama R et al. J Clin Oncol. 2008; 26(26):4244-4252. doi:10.1200/JCO.2007.15.0185; and Mok T et al., N Engl J Med 2009; 361:947-957). Driver mutation tests are therefore required prior to targeted therapy.

Tissue biopsy has been the primary source for mutation identification. However, it is not ideal for NSCLC patients. Approximately 75% of the NSCLC cases are advanced at diagnosis (Reade C et al., Biol targets Ther. 2009:215-224) but solid biopsy has an inherent disadvantage in these cases of detecting intertumoral and intratumoral heterogeneity, which often leads to drug resistance. In fact, the presence of tumor heterogeneity is a major challenge in developing effective cancer treatment using targeted therapies (Yancovitz M et al, PLoS One. 2012; 7(1):e29336. doi:10.1371/journal.pone.0029336). Liquid biopsy as an attractive approach has the potential to capture a comprehensive profile of genomic alterations and thus allows delivery of effective targeted therapy. Moreover, a liquid biopsy is easily repeatable, which makes it possible to monitor the tumor dynamics and, thus, to guide drug changes during therapy. Liquid biopsy can also potentially be used after surgery or therapy to measure minimal residual disease that may result in recurrence (Diehl F et al., Nat Med. 2008; 14(9):985-990. doi:10.1038/nm.1789). In addition, it can be an important companion tool in the follow-up care to detect early signs of relapse (Misale S et al., Nature. 2012; 486(7404):532-536. doi:10.1038/nature11156 and Diaz L a et al., Nature. 2012; 486(7404):537-540. doi:10.1038/nature11219)). However, due to the low presence of circulating tumor nucleic acids (ctNA) and circulating tumor cells, aggravated by artificial errors introduced by detection methods, liquid biopsy has not been used by physicians in daily practice.

The system and method disclosed in this invention can be used as a reliable and rapid liquid biopsy assay for late stage NSCLC patients. The barriers to develop such a reliable and rapid assay are twofold and no currently available platform yet overcomes both.

The first one is sensitivity. The presence of circulating tumor DNA (ctDNA) even in late stage cancer patients can be extremely low. Newman et al observed a range of 0.04% to 3.2% ctDNA in plasma of advanced stage NSCLC patients (Newman A M et al., Nat Med. 2014; 20(5):548-554. doi:10.1038/nm.3519). Quantitative PCR (qPCR), a commonly used technology, can reach limit of detection (LOD) of about 1% and Next Generation Sequencing (NGS) can mostly reach a 1-2% LOD. Digital PCR in this aspect shows the most promising advance, with a low LOD of 0.01% (Detection of rare mutations in blood samples by droplet digital PCR. http://www.bio-rad.com/webroot/web/pdf/lsr/literature/Bulletin_6317.pdf.).

The second is multiplex capability. The number of discovered actionable mutations and the limited volume of blood sample render a compilation of singleplex assays unsuitable for liquid biopsy. Therefore, a crucial feature of a functional liquid biopsy is the ability to examine multiple driver mutations in parallel from a single plasma DNA sample. NGS is the only mature platform currently offering sufficient multiplex capability.

The liquid biopsy disclosed herein focuses on the identification of actionable oncogenic mutations to guide therapy selection. Approximately 75% of the NSCLC are metastatic or advanced at diagnosis (Reade C et al., Biol targets Ther. 2009:215-224) and are eligible for targeted therapy. In these patients tumor heterogeneity is present at a high level. A primary objective here is to test for actionable mutations in a heterogenic tumor cell population and then closely monitor tumor response to therapy and the rise of molecular resistance. Patients under follow-up care post-treatment also greatly benefit from liquid biopsy. Studies have found that molecular tests can detect relapses months before radiologic examination (Misale S et al., Nature. 2012; 486(7404):532-536. doi:10.1038/nature11156 and Diaz L a et al., Nature. 2012; 486(7404):537-540. doi:10.1038/nature11219).

As described in the examples below, the system and method disclosed herein were successfully used to enrich and detect a number of mutations at certain hotspots including those encoding EGFR T790M, EGFR L858R, BRAF V600E, BRAF V600K, BRAF V600D, BRAF V600G, BRAF V600A, and BRAF V600R.

EGFR T790M mutation is a frequently acquired mutation in patients on TKI targeted therapy that results in an amino acid substitution from threonine to methionine at EGFR position 790. This mutated residue increases affinity to ATP and outcompetes the binding of the inhibitors (Yun C-H et al., Proc Natl Acad Sci USA. 2008; 105(6):2070-2075. doi:10.1073/pnas.0709662105). Patients start to show reduced sensitivity to TKI with as low as 5% of cancer cells that acquired this mutation (Lindeman N I et al., Arch Pathol Lab Med. 2013; 137(6):828-860. doi:10.5858/arpa.2012-0720-OA.). Therefore, it is necessary to closely monitor the emergence of T790M. The detection of T790M may assist a physician's decision making of switching drug to the third-generation EGFR TKIs or a hiatus during therapy (Watanabe S et al., BMC Cancer. 2011; 11(1):1. doi:10.1186/1471-2407-11-1).

EGFR L858R is an oncogenic driver that accounts for 43% of all EGFR activated lung cancer (Mitsudomi T et al., FEBS J. 2010; 277(2):301-308. doi:10.1111/j.1742-4658.2009.07448.x). DNA from a cell line containing EGFR L858R mutation was mixed at different ratios with wild type DNA and enriched using the method and system disclosed above (Table 3). Similar to T790M's outcome, the 863-fold enrichment is more than adequate to detect 0.01% mutant alleles.

In addition to the above point mutations, other nucleic acid variants or mutations can be enriched and/or amplified by the method and system of this invention. Examples of the nucleic acid variants include those listed Tables 8 and 9 below.

The system and method disclosed herein provide best-in-class liquid biopsy products. Compared to in silico enhanced liquid biopsies that may take up the full capacity of an Illumina HiSeq by a single biopsy sample (Sullivan M. Guardant Health takes another $50M for “liquid biopsy” cancer test. 2015. http://venturebeat.com/2015/02/03/guardant-health-takes-another-50m-for-ground-breaking-liquid-biopsy-test), the assay disclosed herein reduces the presence of non-mutated DNA in vitro to allow more sensitive detection of the oncogenic mutations and is able to use the same HiSeq sequencing capacity to process 10,000 samples.

### Compositions and Kits

The invention encompasses a composition or reaction mixture comprising the aforementioned primers and blockers. The composition can further comprise one or more reagents selected from the group consisting of a nucleic acid polymerase, extension nucleotides, and a detecting agent.

The detecting agent can be a nucleotide probe, such as a molecular beacon probe or a Yin-Yang probe that is labeled with a fluorophore and a quencher. See e.g., U.S. Pat. Nos. 5,925,517, 6,103,476, 6,150,097, 6,270,967, 6,326,145, and 7,799,522. The composition can also comprise, in addition to the above reagents, one or more of: a salt, e.g., NaCl, MgCl₂, KCl, MgSO₄; a buffering agent, e.g., a Tris buffer, N-(2-Hydroxyethyl)piperazine-N′-(2-ethanesulfonic acid) (HEPES), 2-(N-Morpholino)ethanesulfonic acid (MES), MES sodium salt, 3-(N-Morpholino)propanesulfonic acid (MOPS), N-tris[Hydroxymethyl]methyl-3-aminopro-panesulfonic acid (TAPS); a solubilizing agent; a detergent, e.g., a non-ionic detergent such as Tween-20; a nuclease inhibitor; and the like.

The invention encompasses kits and diagnostic systems for conducting amplification, enrichment, and/or for detection of a target sequence. To that end, one or more of the reaction components for the methods disclosed herein can be supplied in the form of a kit for use in the enrichment and detection of a target nucleic acid strand. In such a kit, an appropriate amount of one or more reaction components is provided in one or more containers or held on a substrate (e.g., by electrostatic interactions or covalent bonding).

A kit containing reagents for performing amplification or enrichment or sequencing (such as those for NGS or Sanger sequencing) of a target nucleic acid sequence using the methods described herein may include one or more of the followings: a forward primer, a reverse primer, one or more blockers, a nucleic acid polymerase, extension nucleotides, and detection probes. Examples of additional components of the kits include, but are not limited to, one or more different polymerases, one or more primers that are specific for a control nucleic acid or for a target nucleic acid, one or more probes that are specific for a control nucleic acid or for a target nucleic acid, buffers for polymerization reactions (in 1× or concentrated forms), and one or more dyes or fluorescent molecules for detecting polymerization products. The kit may also include one or more of the following components: supports, terminating, modifying or digestion reagents, osmolytes, and an apparatus for detecting a detection probe.

The reaction components used in an amplification and/or detection process may be provided in a variety of forms. For example, the components (e.g., enzymes, nucleotide triphosphates, blockers, and/or primers) can be suspended in an aqueous solution or as a freeze-dried or lyophilized powder, pellet, or bead. In the latter case, the components, when reconstituted, form a complete mixture of components for use in an assay.

A kit or system may contain, in an amount sufficient for at least one assay, any combination of the components described herein, and may further include instructions recorded in a tangible form for use of the components. In some applications, one or more reaction components may be provided in pre-measured single use amounts in individual, typically disposable, tubes or equivalent containers. With such an arrangement, the sample to be tested for the presence of a target nucleic acid can be added to the individual tubes and amplification carried out directly. The amount of a component supplied in the kit can be any appropriate amount, and may depend on the target market to which the product is directed. General guidelines for determining appropriate amounts may be found in, for example, Joseph Sambrook and David W. Russell, Molecular Cloning: A Laboratory Manual, 3rd edition, Cold Spring Harbor Laboratory Press, 2001; and Frederick M. Ausubel, Current Protocols in Molecular Biology, John Wiley & Sons, 2003.

The kits of the invention can comprise any number of additional reagents or substances that are useful for practicing a method of the invention. Such substances include, but are not limited to: reagents (including buffers) for lysis of cells, divalent cation chelating agents or other agents that inhibit unwanted nucleases, control DNA for use in ensuring that the enzyme complexes and other components of reactions are functioning properly, DNA fragmenting reagents (including buffers), amplification reaction reagents (including buffers), and wash solutions. The kits of the invention can be provided at any temperature. For example, for storage of kits containing protein components or complexes thereof in a liquid, it is preferred that they are provided and maintained below 0° C., preferably at or below −20° C., or otherwise in a frozen state.

The container(s) in which the components are supplied can be any conventional container that is capable of holding the supplied form, for instance, microfuge tubes, ampoules, bottles, or integral testing devices, such as fluidic devices, cartridges, lateral flow, or other similar devices. The kits can include either labeled or unlabeled nucleic acid probes for use in detection of target nucleic acids. In some embodiments, the kits can further include instructions to use the components in any of the methods described herein, e.g., a method using a crude matrix without nucleic acid extraction and/or purification. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like).

A system, in addition to containing kit components, may further include instrumentation for conducting an assay, e.g. a luminometer for detecting a signal from a labeled probe.

Instructions, such as written directions or videotaped demonstrations detailing the use of the kits or system of the present invention, are optionally provided with the kit or systems. In a further aspect, the present invention provides for the use of any composition or kit herein, for the practice of any method or assay herein, and/or for the use of any apparatus or kit to practice any assay or method herein.

Optionally, the kits or systems of the invention further include software to expedite the generation, analysis and/or storage of data, and to facilitate access to databases. The software includes logical instructions, instructions sets, or suitable computer programs that can be used in the collection, storage and/or analysis of the data. Comparative and relational analysis of the data is possible using the software provided.

All of the above-described methods, reagents, and systems provide a variety of diagnostic tools which permit a blood-based, non-invasive assessment of disease status in a subject. Use of these methods, reagents, and systems in diagnostic tests, which may be coupled with other screening tests, such as a chest X-ray or CT scan, increase diagnostic accuracy and/or direct additional testing. In other aspects, the inventions described herein permit the prognosis of disease, monitoring response to specific therapies, and regular assessment of the risk of recurrence. The inventions described herein also permit the evaluation of changes in diagnostic signatures present in pre-surgery and post therapy samples and identifies a gene expression profile or signature that reflects tumor presence and may be used to assess the probability of recurrence.

A significant advantage of the methods of this invention over existing methods is that they are able to characterize the disease state from a minimally-invasive procedure, i.e., by taking a blood sample without isolating cancer cells. In contrast current practice for classification of cancer tumors from gene expression profiles depends on a tissue sample, usually a sample from a tumor. In the case of very small tumors, a biopsy is problematic and clearly if no tumor is known or visible, a sample from it is impossible. No purification or isolation of tumor is required, as is the case when tumor samples are analyzed. Blood samples have an additional advantage, which is that the material is easily prepared and stabilized for later analysis, which is important when messenger RNA is to be analyzed.

### Definitions

A “nucleic acid” refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA.

As used herein, the term “target nucleic acid” or “target” refers to a nucleic acid containing a target nucleic acid sequence. A target nucleic acid may be single-stranded or double-stranded, and often is DNA, RNA, a derivative of DNA or RNA, or a combination thereof. A “target nucleic acid sequence,” “target sequence” or “target region” means a specific sequence comprising all or part of the sequence of a single-stranded nucleic acid. A target sequence may be within a nucleic acid template, which may be any form of single-stranded or double-stranded nucleic acid. A template may be a purified or isolated nucleic acid, or may be non-purified or non-isolated.

The term “allele” refers generally to alternative DNA sequences at the same physical locus on a segment of DNA, such as, for example, on homologous chromosomes. An allele can refer to DNA sequences which differ between the same physical locus found on homologous chromosomes within a single cell or organism or which differ at the same physical locus in multiple cells or organisms (“allelelic variant”). In some instances, an allele can correspond to a single nucleotide difference at a particular physical locus. In other embodiments and allele can correspond to nucleotide (single or multiple) insertion or deletion.

As used herein, the term “rare allelic variant” refers to a target polynucleotide present at a lower level in a sample as compared to an alternative allelic variant. The rare allelic variant may also be referred to as a “minor allelic variant” and/or a “mutant allelic variant.” For instance, the rare allelic variant may be found at a frequency less than 1/10, 1/100, 1/1,000, 1/10,000, 1/100,000, 1/1,000,000, 1/10,000,000, 1/100,000,000 or 1/1,000,000,000 compared to another allelic variant for a given SNP or gene. Alternatively, the rare allelic variant can be, for example, less than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, or 1,000,000 copies per 1, 10, 100, 1,000 micro liters of a sample or a reaction volume.

As used herein, the terms “abundant allelic variant” may refer to a target polynucleotide present at a higher level in a sample as compared to an alternative allelic variant. The abundant allelic variant may also be referred to as a “major allelic variant” and/or a “wild type allelic variant.” For instance, the abundant allelic variant may be found at a frequency greater than 10×, 100×, 1,000×, 10,000×, 100,000×, 1,000,000×, 10,000,000×, 100,000,000×. or 1,000,000,000× compared to another allelic variant for a given SNP or gene. Alternatively, the abundant allelic variant can be, for example, greater than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, 1,000,000 copies per 1, 10, 100, 1,000 micro liters of a sample or a reaction volume.

As used herein the term “amplification” and its variants includes any process for producing multiple copies or complements of at least some portion of a polynucleotide, said polynucleotide typically being referred to as a “template.” The template polynucleotide can be single stranded or double stranded. Amplification of a given template can result in the generation of a population of polynucleotide amplification products, collectively referred to as an “amplicon.” The polynucleotides of the amplicon can be single stranded or double stranded, or a mixture of both. Typically, the template will include a target sequence, and the resulting amplicon will include polynucleotides having a sequence that is either substantially identical or substantially complementary to the target sequence. In some embodiments, the polynucleotides of a particular amplicon are substantially identical, or substantially complementary, to each other; alternatively, in some embodiments the polynucleotides within a given amplicon can have nucleotide sequences that vary from each other. Amplification can proceed in linear or exponential fashion, and can involve repeated and consecutive replications of a given template to form two or more amplification products. Some typical amplification reactions involve successive and repeated cycles of template-based nucleic acid synthesis, resulting in the formation of a plurality of daughter polynucleotides containing at least some portion of the nucleotide sequence of the template and sharing at least some degree of nucleotide sequence identity (or complementarity) with the template. In some embodiments, each instance of nucleic acid synthesis, which can be referred to as a “cycle” of amplification, includes creating free 3′ end (e.g., by nicking one strand of a dsDNA) thereby generating a primer and primer extension steps; optionally, an additional denaturation step can also be included wherein the template is partially or completely denatured. In some embodiments, one round of amplification includes a given number of repetitions of a single cycle of amplification. For example, a round of amplification can include 5, 10, 15, 20, 25, 30, 35, 40, 50, 75, 100 or more repetitions of a particular cycle. In one exemplary embodiment, amplification includes any reaction wherein a particular polynucleotide template is subjected to two consecutive cycles of nucleic acid synthesis. The synthesis can include template-dependent nucleic acid synthesis.

The term “blocker” (also referred to herein as “oligonucleotide blocker,” “blocking oligo,” “blocker probe,” or “oligo blocker”) refers to a strand of nucleic acid or an oligonucleotide capable of hybridizing to a strand of DNA comprising a particular allelic variant which is located on the same, opposite or complementary strand as that bound by a primer (either a forward primer or a reverse primer), and reduces or prevents amplification of that particular allelic variant. A blocker can be designed, for example, so as to tightly bind to a wild type allele (e.g., abundant allelic variant) in order to suppress amplification of the wild type allele while amplification is allowed to occur on the same or opposing strand comprising a mutant allele (e.g., rare allelic variant) by extension of a primer.

The term “primer” or “primer oligonucleotide” refers to a strand of nucleic acid or an oligonucleotide capable of hybridizing to a template nucleic acid and acting as the initiation point for incorporating extension nucleotides according to the composition of the template nucleic acid for nucleic acid synthesis. “Extension nucleotides” refer to any nucleotide capable of being incorporated into an extension product during amplification, i.e., DNA, RNA, or a derivative if DNA or RNA, which may include a label.

As used herein, the term “modified base” refers generally to any modification of a base or the chemical linkage of a base in a nucleic acid that differs in structure from that found in a naturally occurring nucleic acid. Such modifications can include changes in the chemical structures of bases or in the chemical linkage of a base in a nucleic acid, or in the backbone structure of the nucleic acid. (See, e.g., Latorra, D. et al., Hum Mut 2003, 2:79-85. Nakiandwe, J. et al., Plant Method 2007, 3:2.).

The term “detection probe” refers to an oligonucleotide having a sequence sufficiently complementary to its target sequence to form a probe:target hybrid stable for detection under stringent hybridization conditions. A probe is typically a synthetic oligomer that may include bases complementary to sequence outside of the targeted region which do not prevent hybridization under stringent hybridization conditions to the target nucleic acid. A sequence non-complementary to the target may be a homopolymer tract (e.g., poly-A or poly-T), promoter sequence, restriction endonuclease recognition sequence, or sequence to confer desired secondary or tertiary structure (e.g., a catalytic site or hairpin structure), or a tag region which may facilitate detection and/or amplification. “Stable” or “stable for detection” means that the temperature of a reaction mixture is at least 2° C. below the melting temperature (Tm) of a nucleic acid duplex contained in the mixture, more preferably at least 5° C. below the Tm, and even more preferably at least 10° C. below the Tm.

Hybridization” or “hybridize” or “anneal” refers to the ability of completely or partially complementary nucleic acid strands to come together under specified hybridization conditions in a parallel or preferably antiparallel orientation to form a stable double-stranded structure or region (sometimes called a “hybrid”) in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

“Preferentially hybridize” means that under stringent hybridization conditions, nucleic acids or oligonucleotides (e.g., primers, blockers, or probes) can hybridize to their target nucleic acid sequence to form stable hybrids, e.g., to indicate the presence of at least one sequence or organism of interest in a sample. A nucleic acid hybridizes to its target nucleic acid specifically, i.e., to a sufficiently greater extent than to a non-target nucleic acid to accurately detect the presence (or absence) of the intended target sequence. Preferential hybridization generally refers to at least a 10-fold difference between target and non-target hybridization signals in a sample.

The term “stringent hybridization conditions” or “stringent conditions” means conditions in which a nucleic acid or oligomer hybridizes specifically to its intended target nucleic acid sequence and not to another sequence. Stringent conditions may vary depending well-known factors, e.g., GC content and sequence length, and may be predicted or determined empirically using standard methods well known to one of ordinary skill in molecular biology (e.g., Sambrook, J. et al., 1989, Molecular Cloning, A Laboratory Manual, 2nd ed., Ch. 11, pp. 11.47-11.57, (Cold Spring Harbor Laboratory Press, Cold Spring Harbor, N.Y.)).

“Substantially homologous” or “substantially corresponding” means a probe, nucleic acid, or oligonucleotide has a sequence of at least 10, 20, 30, 40, 50, 100, 150, 200, 300, 400, or 500 contiguous bases that is at least 80% (preferably at least 85%, 90%, 95%, 96%, 97%, 98%, and 99%, and most preferably 100%) identical to contiguous bases of the same length in a reference sequence. Homology between sequences may be expressed as the number of base mismatches in each set of at least 10 contiguous bases being compared.

“Substantially complementary” means that an oligonucleotide has a sequence containing at least 10, 20, 30, 40, 50, 100, 150, 200, 300, 400, or 500 contiguous bases that are at least 80% (preferably at least 85%, 90%, 95%, 96%, 97%, 98%, and 99%, and most preferably 100%) complementary to contiguous bases of the same length in a target nucleic acid sequence. Complementarity between sequences may be expressed a number of base mismatches in each set of at least 10 contiguous bases being compared.

As used herein, the term “subject” refers to any organism having a genome, preferably, a living animal, e.g., a mammal, which has been the object of diagnosis, treatment, observation or experiment. Examples of a subject can be a human, a livestock animal (beef and dairy cattle, sheep, poultry, swine, etc.), or a companion animal (dogs, cats, horses, etc).

The term “biological sample” refers to a sample obtained from an organism (e.g., patient) or from components (e.g., cells) of an organism. The sample may be of any biological tissue, cell(s) or fluid. The sample may be a “clinical sample” which is a sample derived from a subject, such as a human patient or veterinary subject. Such samples include, but are not limited to, saliva, sputum, blood, blood cells (e.g., white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample.” A biological sample may also include a substantially purified or isolated protein, membrane preparation, or cell culture.

As used herein, the term “contacting” and its variants, when used in reference to any set of components, includes any process whereby the components to be contacted are mixed into same mixture (for example, are added into the same compartment or solution), and does not necessarily require actual physical contact between the recited components. The recited components can be contacted in any order or any combination (or subcombination), and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting a template with a reaction mixture” includes any or all of the following situations: (i) the template is contacted with a first component of the reaction mixture to create a mixture; then other components of the reaction mixture are added in any order or combination to the mixture; and (ii) the reaction mixture is fully formed prior to mixture with the template.

The term “mixture” as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not addressable. To be specific, an array of surface-bound oligonucleotides, as is commonly known in the art and described below, is not a mixture of surface-bound oligonucleotides because the species of surface-bound oligonucleotides are spatially distinct and the array is addressable.

By “diagnosis” or “evaluation” of a cancer (e.g., a lung cancer or melanoma) refers to a diagnosis of a cancer, a diagnosis of a stage of the cancer, a diagnosis of a type or classification of the cancer, a diagnosis or detection of a recurrence of the cancer, a diagnosis or detection of a regression of the cancer, a prognosis of the cancer, or an evaluation of the response of the cancer to a surgical or non-surgical therapy.

Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other diagnosing and/or staging methods known in the medical art for a particular disease or disorder, e.g., lung cancer or melanoma.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 20” may indicate a range of 18 to 22, and “about 1” may mean from 0.9-1.1. Other meanings of “about” may be apparent from the context, such as rounding off, so, for example “about 1” may also mean from 0.5 to 1.4.

Preferred embodiments are illustrated herein, but those skilled in the art will appreciate that other components and conditions in addition to those illustrated may be used in the methods described herein.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated herein in their entireties.

### EXAMPLES

### Example 1

In this example, three mutation hotspots, EGFR T790M, EGFR L858R, and BRAF V600E/V600K/V600D/V600G/V600A/V600R, served as a model system to practice the method disclosed above. Several types of PCR chemistries and nucleic acid modifications were tested. The results described below demonstrated the feasibility of the enrichment system disclosed herein.

Listed below are blockers used.

TABLE 1A

| | | SEQ |
|---|---|---|
| | | ID |
| Name | Sequences | No |
| EGFR-T790M- | ACCGTGCAACTCATCA* + C | 1 |
| probe-F | | |
| EGFR-T790M- | GGCATGAGCTGC* + G | 2 |
| probe-R | | |
| EGFR-T790M-LR | AGGGCATGAGCTGC* + G | 3 |
| EGFR-T790M-2-LR | AGGGCATGAGCTG + C*G | 4 |
| EGFR-L858R- | GATCACAGATTTTGGGCT | 5 |
| probe-F | | |
| EGFR-L858R- | CCAGCAGTTTGGCCA | 6 |
| probe-R | | |
| EGFR-L858R-2-F | GATCACAGATTTTGGG + C*T | 7 |
| EGFR-L858R-2-R | CAGCAGTTTGGC + C*A | 8 |
| EGFR-19del-F-4 | CCGTCGCTATCAAGGAATTAAGAGA + A*G | 9 |
| KRAS-G12-34-R3 | CTTGCCTACGCCA + C*C | 10 |
| KRAS-G12-35-F3 | TGGTAGTTGGAGCT + G*G | 11 |
| BRAF-V600-F | ATTTTGGTCTAGCTACAC + T | 12 |
| BRAF-V600-R | CCACTCCATCGAGATTTGA | 13 |
| BRAF-V600-2-F | TGATTTTGGTCTAGCTACA + G*T | 14 |
| BRAF-V600-2-R | CCACTCCATCGAGATTT + C*A | 15 |

*phosphorothioate bond
+ LNA

Listed below are primers used

TABLE 1B

| | | SEQ |
|---|---|---|
| | | ID |
| Name | Sequences | No |
| EGFR_e19_2-P5 | ACACTCTTTCCCTACACGACGCTCTTCCG | 16 |
| | ATCTGTCATAGGGACTCTGGATCCCA | |
| EGFR_e19_2-P7 | GACTGGAGTTCAGACGTGTGCTCTTCCGA | 17 |
| | TCTCCACACAGCAAAGCAGAAACTC | |
| EGFR_e20-P5 | ACACTCTTTCCCTACACGACGCTCTTCCG | 18 |
| | ATCTCTCCAGGAAGCCTACGTGAT | |
| EGFR_e20-P7 | GACTGGAGTTCAGACGTGTGCTCTTCCGA | 19 |
| | TCTGTCTTTGTGTTCCCGGACAT | |
| EGFR_e21-P5 | ACACTCTTTCCCTACACGACGCTCTTCCG | 20 |
| | ATCTGGAACGTACTGGTGAAAACACC | |
| EGFR_e21-P7 | GACTGGAGTTCAGACGTGTGCTCTTCCGA | 21 |
| | TCTTGCCTCCTTCTGCATGGTATTC | |
| BRAF_e15-P5 | ACACTCTTTCCCTACACGACGCTCTTCCG | 22 |
| | ATCTTTCATGAAGACCTCACAGTAAAAA | |
| BRAF_e15-P7 | GACTGGAGTTCAGACGTGTGCTCTTCCGA | 23 |
| | TCTCCACAAAATGGATCCAGACA | |
| KRAS_e1-P5 | ACACTCTTTCCCTACACGACGCTCTTCCG | 24 |
| | ATCTAGGCCTGCTGAAAATGACTG | |
| KRAS_e1-P7 | GACTGGAGTTCAGACGTGTGCTCTTCCGA | 25 |
| | TCTTTGTTGGATCATATTCGTCCAC | |

EGFR T790M mutation is a frequently acquired mutation in patients on TKI targeted therapy that results in an amino acid substitution from threonine to methionine at EGFR position 790. This mutated residue increases affinity to ATP and outcompetes the binding of the inhibitors. Patients start to show reduced sensitivity to TKI with as low as 5% of cancer cells that acquired this mutation. Therefore, it is necessary to closely monitor the emergence of T790M. The detection of T790M may assist a physician's decision making of switching drug to the third-generation EGFR TKIs or a hiatus during therapy.

DNA from a cell line containing EGFR T790M mutation was mixed at different ratios with wild type DNA and enriched (Table 2). The resulting libraries were sequenced on Illumina MiSeq, and the sequence data were processed on CLC Genomics Workbench. The enrichment fold increases as the sample's mutant concentration lowers due to less physical restriction by the increased amount of mutant alleles that cannot exceed 100%. It was found that the method and system disclosed above were able to enrich T790M from a sample of 0.015% mutant content to 8.5%, a 583-fold enrichment, and confidently calls the variant.

TABLE 2
EGFR T790M enrichment result

| | before enrichment % | after enrichment % | enrichment fold |
|---|---|---|---|
| EGFR | 1.5 | 68.5 | 47 |
| T790M | 0.15 | 15.4 | 106 |
| | 0.015 | 8.5 | 583 |

EGFR L858R is an oncogenic driver that accounts for 43% of all EGFR activated lung cancer. DNA from a cell line containing EGFR L858R mutation was mixed at different ratios with wild type DNA and enriched using the method and system disclosed above (Table 3). Similar to T790M's outcome, the 863-fold enrichment is more than adequate to detect 0.01% mutant alleles.

TABLE 3
EGFR L858R enrichment result

| | before enrichment % | after enrichment % | enrichment fold |
|---|---|---|---|
| EGFR | 0.5 | 83.0 | 148 |
| L858R | 0.1 | 50.3 | 450 |
| | 0.02 | 19.3 | 863 |

Since blockers were designed based on the wild type sequences, the method and system disclosed above can enrich more than one type of mutation at any particular locus. For instance, at BRAF Valine 600, the method and system disclosed above have the capability to enrich and detect V600E, V600K, V600D, V600G, V600A, and V600R mutations via a single pair of blockers. Table 4 demonstrates that the BRAF V600 blockers enriched, in addition to V600E, all BRAF 600 mutations simultaneously from a mixture of three BRAF mutants in a wild type background.

TABLE 4
Enrichment result of BRAF Valine 600 mutation mixture
BRAF V600

| before | enriched | enrich- | enriched | enrich- | enriched | enrich- |
|---|---|---|---|---|---|---|
| enrich- | % | ment | % | ment | % | ment |
| ment %* | V600E | fold | V600G | fold | V600R | fold |
| 10 | 65.0 | 9 | 14.8 | 13 | 12.5 | 12 |
| 1 | 71.9 | 96 | 7.2 | 64 | 8.9 | 82 |
| 0.1 | 25.6 | 342 | 6.0 | 536 | 2.7 | 247 |

*% of total mutation in wild type background, individual allele concentration varies

To demonstrate reproducibility, eight 0.01% EGFR T790M enrichment replicates were processed (Table 5) and the enrichment ranges 4.1-7.9%, less than a twofold difference. These results are distinctly higher than artificial mutations enriched in wild type due to nucleotide misincorporation (up to 1.2%) and thus variant calling is not affected. To detect mutant from <0.01% samples, a confident threshold can be determined as described below. The concentrations prior to and after enrichment have a monotonic increasing relationship that closely resembles a logarithmic function. Given that the enrichment outcome is highly reproducible, one can use the logarithmic relationship to estimate a range of concentration in which the ctDNA is present prior to enrichment.

TABLE 5
Eight replicates of T790M enrichment from a 0.01% sample

| | Rxn | Rxn | Rxn | Rxn | Rxn | Rxn | Rxn | Rxn |
|---|---|---|---|---|---|---|---|---|
| | 1 | 2 | 3 | 4 | 5 | 6 | 7 | 8 |
| after enrichment % | 6.9 | 6.1 | 4.7 | 4.4 | 4.1 | 4.4 | 4.7 | 7.9 |
| enrichment fold | 627 | 555 | 426 | 398 | 376 | 396 | 431 | 717 |

The method disclosed herein can be designed to be easily multiplexed, and here it was shown that all three sites above can be co-enriched (Table 6). Accordingly, the method disclosed herein not only allows one to build presently a multiplex panel detecting the mutations listed in Table 8, but also to cover more mutations as their clinical utilities are demonstrated in the future.

TABLE 6
Triplex enrichment result

| | before | after | enrich- | before | after | enrich- |
|---|---|---|---|---|---|---|
| | enrich- | enrich- | ment | enrich- | enrich- | ment |
| Blockers | ment % | ment % | fold | ment % | ment % | fold |
| EGFR | 0.5 | 40.3 | 77 | 0.05 | 18.6 | 357 |
| T790M | | | | | | |
| EGFR | | 92.1 | 184 | | 29.5 | 590 |
| L858R | | | | | | |
| BRAF | | 90.6 | 174 | | 70.0 | 1346 |
| V600E | | | | | | |

### Example 2

RNA is a more efficient source for detecting various kinase fusions. An ALK or ROS1 fusion transcript can be resulted from a plurality of chromosomal rearrangements joining at introns. A cell line expressing a SLC34A2-ROS1 fusion was tested. RNA was reversed transcribed and diluted up to 100-fold with wild type cDNA, and in all cases, the fusion was confidently called.

In summary, the above results demonstrate that the system and method disclosed herein can confidently detect the presence of 0.01% mutation with high reproducibility and the system is capable of examining multiple mutation sites in parallel.

TABLE 7
EGFR T790M enrichment improvements

| before | | after | enrichment |
|---|---|---|---|
| enrichment % | Blocker set | enrichment % | fold |
| 0.01 | T790M set 1 | 0.1 | 13 |
| | T790M set 2 | 1.6 | 146 |
| | T790M set 3 | 1.8 | 167 |
| | T790M set 4 | 2.1 | 201 |
| | T790M set 5 | 7.8 | 711 |

### Example 3

Assays are carried out to design and test primers and blockers for mutations occurring in ≧1% NSCLC patients for which an approved therapy exists.

Similar to primers, the blockers at each locus are empirically verified but often require further optimization. Inventors have discovered a number of ways to adjust blocker's composition and, thus, optimize the blocking efficiency. Table 7 shows the performance of each EGFR T790M blocker set and how the adjustments can improve the enrichment efficiency.

Table 8 lists the actionable driver mutations with clear clinical utilities. Each mutation is assayed individually in singleplex reactions, where cell line DNA harboring each of these mutations is titrated to 1%, 0.1%, and 0.01% and used for enrichment. Mutant and wild type DNA is quantified by Qubit, a concentration of 1% sample is confirmed using qPCR and Illumina MiSeq sequencing. Subsequent 0.1% and 0.01% serial dilutions are performed from 1% sample.

TABLE 8
List of actionable mutations to be enriched

| | | | |
|---|---|---|---|
| EGFR c.2155 | EGFR | EGFR | EGFR |
| | c.2238_2252del15 | c.2240_ | c.2369 |
| | | 2257del18 | |
| EGFR c.2156 | EGFR c.2239_2247 | EGFR c.2307_ | EGFR |
| | delTTAAGAGAA | 2308 | c.2573 |
| | | insGCCAGCGTG | |
| EGFR | EGFR c.2239_2248 | EGFR c.2308_ | EGFR |
| c.2235_ | TTAAGAGAAG > C | 2309 | c.2582 |
| 2246del12 | (SEQ ID NO: 26) | insCCAGCGTGG | |
| EGFR | EGFR | EGFR | BRAF |
| c.2235_ | c.2239_2251 > C | c.2231_ | c.1799 |
| 2249del15 | | 2232ins18 | |
| EGFR | EGFR | EGFR | KRAS |
| c.2236_ | c.2239_2253del15 | c.2234_ | c.34 |
| 2250del15 | | 2235ins18 | |
| EGFR | EGFR | EGFR | KRAS |
| c.2237_ | c.2239_2256del18 | c.2236_ | c.35 |
| 2251del15 | | 2237ins18 | |
| EGFR | EGFR | EGFR | KRAS |
| c.2237_ | c.2239_2257 > GT | c.2232_ | c.37 |
| 2253 > TTGCT | | 2233ins18 | |
| EGFR c.2237_ | EGFR | EGFR c.2303 | KRAS |
| 2255 > T | c.2240_2254del15 | | c.38 |

### Example 4

In this example, assays are carried out to multiplex the enrichment and test compatibility.

The primer pool are first tested without the presence of blockers and optimized for uniform amplification. A list of mutations that can co-exist in NSCLC patients is compiled and used to guide multiplex test. Cell line DNA containing these compatible mutations is mixed. Multiplex enrichment reactions are performed on 1%, 0.1%, and 0.01% DNA mixtures representing the levels of ctDNA in blood. All mutation sites are co-enriched.

TABLE 9
List of ALK and ROS1 fusion junctions

| | | |
|---|---|---|
| EML4_E13-ALK-E20 | EML4_E14; del12- | SLC34A2_E13- |
| | ALK-E20 | ROS1_E32 |
| EML4_E20-ALK-E20 | KIF5B_E24- | GOPC_E8- |
| | ALK_E20 | ROS1_E35 |
| EML4_E6-ALK-E20 | KIF5B_E15- | GOPC_E4- |
| | ALK_E20 | ROS1_E36 |
| EML4_E6; ins33-ALK- | KIF5B_E17- | EZR_E10-ROS1_E34 |
| E20 | ALK_E20 | |
| EML4_E14; ins11del49- | TFG_E4- | SDC4_E2-ROS1_E32 |
| ALK-E20 | ALK_E20 | |
| EML4_E2-ALK-E20 | CD74_E6- | SDC4_E4-ROS1_E32 |
| | ROS1_E34 | |
| EML4_E2; ins117- | CD74_E6- | TPM3_E8- |
| ALK-E20 | ROS1_E32 | ROS1_E35 |
| EML4_E13; ins69- | SLC34A2_E4- | LRIG3_E16- |
| ALK-E20 | ROS1_E32 | ROS1_E35 |

### Example 5

In this example, assays are carried out to enrich target regions having frequent fusion junctions.

Primers are designed against frequent junctions (Table 9) of ALK and ROS1 fusion transcripts. Tests are performed on cell line cDNA harboring these junctions. The primers are first tested for amplification efficiency, then cDNA are diluted with wild type cDNA at different ratios and the minimum amount of equivalent cells needed are determined for confident amplification.

### Example 6

Initial enrichment tests on wild type DNA showed presence of up to 1.2% T790M mutant alleles, augmented errors by nucleotide incorporation. Nonetheless, the elevated error rate observed at EGFR 790 is much less pronounced at EGFR 858 and BRAF 600. Extensive tests are performed to determine confidence threshold for each blocker sets. 50 wild type cell lines in multiple replicates are used to serve as negative controls to determine the range of artificial errors at each locus.

Cell free DNA (cfDNA) standard are generated to test the panel. Briefly, the above mutant DNA titration are sheared by sonication to ˜160 bp resembling cfDNA and tested to estimate the cfDNA quantity needed for reliable detection at each titration. Cell free RNA standard are generated and tested in a similar way.

Initial validation of the liquid biopsy is performed on twenty de-identified NSCLC patient samples obtained from BioServe. Samples are paired specimens of plasma prior to surgery or treatment and FFPE tumor tissue. Also included in BioServe sample collection are five cases of acquired resistance from targeted therapy.

Blockers for driver mutations listed in Table 8 are individually tested to reach a minimum of 100-fold enrichment. Then, they are tested for multiplex enrichment. Primers are pooled and optimized for uniform amplification. Coverage uniformity, defined as total bases present at >0.2× mean coverage, of 90% or higher are reached. Blocker pools are then be tested in the multiplex enrichment PCR for possible oligo interference among amplicons. Problematic primers/blockers are subject to adjustment. Primers to amplify the listed fusion junctions in Table 9 are tested to ensure optimal detection. Variant calling threshold for each of the 56 mutations are individually determined. Patient samples are processed to determine concordance between the paired tissue and blood samples.

Based on the preliminary data and the understanding of the PCR and blocking chemistry, all these mutation sites can be co-enriched. However, if blockers/primers incompatibility occurs, the reactions can be split into two. A list of mutations that can co-exist in NSCLC patients can be compiled, and at a minimum, the mutations that can co-exist can be co-enriched.

Occurrence of cross-contamination and false positives should be rare, yet possible. Because of the simple workflow and low cost, one can construct two libraries in parallel from the same patient sample to minimize, if not eliminate, the rare occasions of contamination and false positives.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A set of nucleic acid molecules for enriching an allelic variant in a target sequence of a target nucleic acid, comprising
a forward primer and a reverse primer that are capable of amplifying the target sequence and
a first blocker comprising a first sequence that (i) is matched or complementary to the wild-type allele in the target sequence and (ii) is capable of being extended by a DNA polymerase.

2. The set of nucleic acid molecules of claim 1, further comprising a second blocker having a second sequence that is matched or complementary to the complement of the wild-type allele.

3. The set of nucleic acid molecules of claim 1, wherein the first or second blocker does not overlap with either the forward primer or the reverse primer.

4. The nucleic acid molecules of claim 1, wherein the first or second blocker contains one or more modified nucleic acids or linkages.

5. The nucleic acid molecules of claim 4, wherein the first or second blocker has a modified nucleic acid or linkage at the 3′ end.

6. The nucleic acid molecules of claim 4, wherein said modified nucleotides or linkages comprise PNA, LNA, a 2′-O-Methyl nucleic acid, a 2′-O-Alkyl nucleic acid, a 2′-fluoro nucleic acid, a phosphorothioate linkage, and any combination thereof.

7. The nucleic acid molecules of claim 1, wherein the target sequence spans a region encoding EGFR T790M, EGFR L858R, BRAF V600E, BRAF V600K, BRAF V600D, BRAF V600G, BRAF V600A, BRAF V600R or one selected from a group consisting of those listed in Tables 8 and 9.

8. The nucleic acid molecules of claim 1, wherein forward primer is about 10 to 50 nucleotides in length, the reverse primer is about 10 to 50 nucleotides in length, and the first or second blocker is about 5 to 100 nucleotides in length

9. A kit comprising the set of nucleic acid molecules of claim 1 and one or more reagents for conducting an amplification reaction.

10. The kit of claim 9, comprising one or more reagents selected from the group consisting of a buffer, a DNA polymerase, an RNAse inhibitor, extension nucleotides, and a probe.

11. A method for enriching an allelic variant in a target sequence comprising
providing the set of nucleic acid molecules of claim 1;
amplifying the target sequence with the forward primer and the reverse primer in the presence of the first or second blocker.

12. A method for detecting the presence or absence of an allelic variant in a target sequence comprising
enriching the allelic variant according to the method of claim 11 to generate a amplification product; and
examining the amplification product for the presence or absence of the allelic variant.

13. The method of claim 12, wherein the examining step is carried out by gene sequencing or qPCR.

14. The method of claim 11, wherein the target sequence spans a region encoding EGFR T790M, EGFR L858R, BRAF V600E, BRAF V600K, BRAF V600D, BRAF V600G, BRAF V600A, BRAF V600R, or one selected from a group consisting of those listed in Tables 8 and 9.

15. A method for evaluating a subject having cancer, comprising
obtaining a biological sample from the subject; and
performing an assay to determine the presence or absence of one or more allelic variants in the biological sample according to the method of claim 14.

16. The method of claim 15, wherein the cancer is lung adenocarcinoma.

17. The method of claim 15, wherein the lung adenocarcinoma is non-small cell lung cancer.

18. The method of claim 15, wherein the biological sample is serum, plasma, whole blood, saliva, or sputum.

19. The method of claim 15, further comprising determining or recommending a treatment course of action based on the presence of said one or more allelic variants.

20. The method of claim 19, further comprising the step of administering said treatment when said one or more allelic variants are present.


SEQUENCE LISTING (26 sequences)

SEQ ID NO: 1 - DNA, 17 bp, Artificial Sequence
  blockers
accgtgcaactcatcac

SEQ ID NO: 2 - DNA, 13 bp, Artificial Sequence
  blocker
ggcatgagctgcg

SEQ ID NO: 3 - DNA, 15 bp, Artificial Sequence
  blocker
agggcatgagctgcg

SEQ ID NO: 4 - DNA, 15 bp, Artificial Sequence
  blocker
agggcatgagctgcg

SEQ ID NO: 5 - DNA, 18 bp, Artificial Sequence
  blocker
gatcacagattttgggct

SEQ ID NO: 6 - DNA, 15 bp, Artificial Sequence
  blocker
ccagcagtttggcca

SEQ ID NO: 7 - DNA, 18 bp, Artificial Sequence
  blocker
gatcacagattttgggct

SEQ ID NO: 8 - DNA, 14 bp, Artificial Sequence
  blocker
cagcagtttggcca

SEQ ID NO: 9 - DNA, 27 bp, Artificial Sequence
  blocker
ccgtcgctatcaaggaattaagagaag

SEQ ID NO: 10 - DNA, 15 bp, Artificial Sequence
  blocker
cttgcctacgccacc

SEQ ID NO: 11 - DNA, 16 bp, Artificial Sequence
  blocker
tggtagttggagctgg

SEQ ID NO: 12 - DNA, 19 bp, Artificial Sequence
  blocker
attttggtctagctacact

SEQ ID NO: 13 - DNA, 19 bp, Artificial Sequence
  blocker
ccactccatcgagatttga

SEQ ID NO: 14 - DNA, 21 bp, Artificial Sequence
  blocker
tgattttggtctagctacagt

SEQ ID NO: 15 - DNA, 19 bp, Artificial Sequence
  blocker
ccactccatcgagatttca

SEQ ID NO: 16 - DNA, 55 bp, Artificial Sequence
  Primer
acactctttccctacacgacgctcttccgatctgtcatagggactctggatccca

SEQ ID NO: 17 - DNA, 54 bp, Artificial Sequence
  Primer
gactggagttcagacgtgtgctcttccgatctccacacagcaaagcagaaactc

SEQ ID NO: 18 - DNA, 53 bp, Artificial Sequence
  Primer
acactctttccctacacgacgctcttccgatctctccaggaagcctacgtgat

SEQ ID NO: 19 - DNA, 52 bp, Artificial Sequence
  Primer
gactggagttcagacgtgtgctcttccgatctgtctttgtgttcccggacat

SEQ ID NO: 20 - DNA, 55 bp, Artificial Sequence
  Primer
acactctttccctacacgacgctcttccgatctggaacgtactggtgaaaacacc

SEQ ID NO: 21 - DNA, 54 bp, Artificial Sequence
  Primer
gactggagttcagacgtgtgctcttccgatcttgcctccttctgcatggtattc

SEQ ID NO: 22 - DNA, 57 bp, Artificial Sequence
  Primer
acactctttccctacacgacgctcttccgatctttcatgaagacctcacagtaaaaa

SEQ ID NO: 23 - DNA, 52 bp, Artificial Sequence
  Primer
gactggagttcagacgtgtgctcttccgatctccacaaaatggatccagaca

SEQ ID NO: 24 - DNA, 53 bp, Artificial Sequence
  Primer
acactctttccctacacgacgctcttccgatctaggcctgctgaaaatgactg

SEQ ID NO: 25 - DNA, 54 bp, Artificial Sequence
  Primer
gactggagttcagacgtgtgctcttccgatctttgttggatcatattcgtccac

SEQ ID NO: 26 - DNA, 11 bp, Homo sapiens
ttaagagaagc